(19) United States
(12) Patent Application Publication
(10) Pub. No.: US 2026/0315218 A1
(43) Pub. Date: Oct. 15, 2026

(54) IMPROVED VALVE INCORPORATING CONSTRUCTED TISSUE

(71) Applicant: VASCUDYNE, INC., St. Paul, MN (US)

(72) Inventors: Richard F. Murphy, White Bear Township, MN (US); Zeeshan Syedain, Stillwater, MN (US); Kemal Schankereli, Stillwater, MN (US)

(21) Appl. No.: 19/112,051

(22) PCT Filed: Sep. 15, 2023
(86) PCT No.: PCT/US2023/032894

§ 371 Date: Mar. 14, 2025

Related U.S. Application Data
(60) Provisional application No. 63/375,837, filed on Sep. 15, 2022.

(51) Int. Cl. A61F 2/24; A61L 27/26; A61L 27/36; A61L 27/54

(52) U.S. Cl. CPC A61F 2/2418; A61F 2/2415; A61L 27/26; A61L 27/3633; A61L 27/3679; A61L 27/54; A61F 2230/0006; A61F 2240/001; A61F 2250/0039; A61L 2300/254; A61L 2300/412; A61L 2300/64

(57) ABSTRACT

The present invention is an implant suitable for use as a valve, typically a heart valve. The implant includes a constructed tissue component that is used to form both a leaflet and a portion of the skirt. Processes for making valve are disclosed.

### CROSS-REFERENCE TO RELATED APPLICATION

This application is a National Phase application under 35 U.S.C. 371 and claims the benefit of International Application No.

PCT/US2023/032894 filed on Sep. 15, 2021, which claims the benefit of U.S. Provisional Application No. 63/375,837 filed Sep. 15, 2022, the disclosures of which are incorporated herein by reference.

### FIELD OF THE INVENTION

The invention relates to products formed from proprietary regenerative tissue, products made from the tissue, an implant comprising this tissue, and methods of treating conditions and/or disorders using such tissue.

### BACKGROUND OF THE INVENTION

A relatively new field of medicine—since the early 1990s—is the field of Regenerative Medicine. Regenerative Medicine is the process of creating functional tissues to repair, replace, or restore tissue or organ structure and function lost due to age, disease, damage, or congenital defects. This field of medicine uses new methods and products including (stem) cell therapy, development of medical devices, and tissue engineering.

The use of prepared heterogenous graft material for human surgical implantation is well known. More specifically, the use of treated animal tissue as human tissue grafts, replacement valves, and similar implantation surgical procedures is well known. However, problems of immunogenicity, thrombogenicity, calcification, material strength, and size have not been adequately addressed in the prior art.

Since the 1930's, medical researchers have attempted to develop suitable natural and synthetic alternatives for obtaining small diameter grafts useful in vascular surgery. Historically, attempts to fabricate such tubular grafts from man-made materials have been somewhat unsuccessful. Homologous tissues are not always readily available and are not always readily available in the size the surgeon needs.

Furthermore, some of these tissues may be immunogenic and therefore may require processing or certain treatments to reduce their immunogenicity.

A variety of materials are available, but all have shortcomings, and none are regenerative. There remains a need for a solution that combines the benefits of native collagenous tissue having regenerative properties with those of manufacturing at scale and in the proper form (i.e., conduit).

Additionally, current artificial valves are static in size and do not grow or adjust to growing bodies. As such, children and adolescents suffering from valvular diseases require multiple procedures to replace artificial valves with larger valves to compensate for the recipient's growth. Since multiple procedures are required as children and adolescents grow, risks and dangers inherent to replacement processes increase with these individuals.

Further, because of the drawbacks associated with conventional open-heart surgery, percutaneous and minimally-invasive surgical approaches are garnering intense attention. In one technique, a prosthetic valve is configured to be implanted in a much less invasive procedure by way of catheterization. For instance, U.S. Pat. Nos. 5,411,522 and 6,730,118, which are incorporated herein by reference in their entireties, describe collapsible transcatheter heart valves that can be percutaneously introduced in a compressed state on a catheter and expanded in the desired position by balloon inflation or by utilization of a self-expanding frame or stent.

A surgical transcatheter valve is also described in Syedain, et al., Sci Trans Med 2021. The surgical implant requires a bottom edge/skirt for sewing the valve into the native anatomy. Leaflets made from the tissue tube can only be closed using a sutured stitch line. This design also leaves a free edge at bottom to be sewn into the native artery. Both stitch lines are in high stress areas, stitch lines that have been shown to degrade fail.

In the present invention, in contrast, the folded tissue technique as disclosed is not practical for surgical valve design. The folded tissue at the bottom permits creating a leaflet with no stress being directly transferred to suture stiches (in prior art designs); the lack of stitches also promotes or maintains greater leaflet durability. Further, a surgical valve does not require any crimping and therefore there is no practical data existing for whether non-fixed engineered tissue can maintain mechanical stability after crimping. In contrast, several of the Examples below are believed to be the first indication of the effect of crimping on a constructed tissue's mechanical properties, e.g., the eighteen-month valve performance proves maintenance of mechanical properties of the leaflets after crimping. Further, the eighteen-month use in vivo as a transcatheter heart valve demonstrates durability of constructed tissue after crimping.

U.S. Pat. No. 11,517,428 (Shang et al.) discloses a TPV valve that uses traditional suture lines to attach the leaflets and the traditional configuration of a skirt separate from the leaflets.

Notwithstanding the usefulness of the above-described methods, a need still exists for increasing patency; making an implant less thrombotic in structure and/or function; minimizing calcification; and increasing the useful life of the implant.

### SUMMARY OF THE INVENTION

One embodiment of the invention is the preparation and use of constructed tissue (CT) to make implants containing one or more valves, their use as implants, and their use in mediating treatment or therapy.

Another embodiment of the invention is the method of making an implantable valve. In this embodiment, tubes formed from constructed tissue (CT), the number of which correspond to the number of leaflets, with a portion of each tube folded to form a skirt.

An advantage of both embodiments of the invention is the starting material itself.

The products, uses, and processes of the present invention are suitable for treating diseases and conditions that would benefit from regenerative engineered tissues, especially those involving tubular tissue constructs.

The present disclosure is directed toward methods and apparatuses relating to prosthetic valves, such as heart valves. An embodiment of the invention includes a tubular engineered tissue device with characteristics suitable for use as a heart valve, more specifically a transcatheter heart valve.

Some embodiments of the invention include the manufacture of a graft that is durable and has been demonstrated in both animal and human trials to withstand the rigorous mechanical requirements of the vascular system.

The biological materials according to the present invention, are processed to modify (e.g., reduce or eliminate) size and shape, thinness, collagen content, and other characteristics and properties that will become clear from the description of the invention. The methods, uses, and products of the present invention are intended for implant in a mammal, preferably a human. All of the biological materials, processed according to the present invention, are appropriate for use in an in vivo environment, and include one or more of the following desirable properties for graft material suitable for implantation: a) size compatibility with surrounding vessels to which it will be anastomosed; b) suturability, kink resistance, softness, radial and longitudinal compliance, and flexibility (a softer hand); c) non-thrombogenicity or low levels of thrombogenicity, particularly after regeneration or recellularization; d) durability; e) ease of sterilization; f) readily available, and available in diameters and lengths appropriate for surgical procedures; g) shelf life appropriate for market conditions (typically greater than three years); h) resistant to infection; i) sufficient strength to resist aneurysm formation; j) non-immunogenic; k) resistant to degradation; I) resistant to formation of neointimal hyperplasia; m) tactile, as expressed by surgeons using the tissue and/or grafts of the present invention, particularly suitable for microsurgery; n) is anti-bacterial; o) is non-infectious; p) is anti-microbial; and q) under circumstances when the implant becomes infected, the implant of the present invention may not become infected or resists infection (e.g., the host/recipient cells become infected exclusive of the implant); r) reduced or eliminated calcification characteristics; s) improved or biologically appropriate or desirable resorbability; t) becomes or transitions into living tissue; and u) in some embodiments, capable of endothelialization. As used herein, living tissue refers to a tissue that exhibits the presence of active cells (originating from the implant recipient), e.g., including but not limited to producing elastin, producing ECM; and/or an un-paralleled lack of immunogenicity.

Some embodiments of the present invention may exhibit one or more valve specific properties including but not limited to somatic growth; maintaining forward flow without increasing pressure gradient over time (age of implant); maintaining valve closing without increased regurgitation; and maintaining effective orifice area (as defined in ISO 5840).

Although specific advantages have been enumerated above, various embodiments may include some, none, or all of the enumerated advantages.

Other technical advantages may become readily apparent to one of ordinary skill in the art after review of the following figures and description.

It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings described below.

With the following enabling description of the drawings, the apparatus should become evident to a person of ordinary skill in the art.

### BRIEF DESCRIPTION OF THE FIGURES

FIG. 1 shows an illustrative process for making an implantable transcatheter valve of the present invention.

FIG. 2 shows how a tube of tissue is cut and configured into a leaflet and skirt.

FIG. 3 shows a side view of the prior art method of stitching a leaflet to a frame.

FIG. 4 shows a bottom view of a typical stitch pattern for a pericardium tissue valve (4A), the tissue valve of Syedain et al cited above (4C), and the lack of stitches in a valve of the present invention (4B).

FIG. 5 is a graph showing the hemodynamic properties of a valve made with constructed tissue mounted inside a nitinol stent over 18 months while implanted in a juvenile sheep model.

FIG. 6 is a graph showing the amount of regurgitation of a valve made with constructed tissue mounted inside a nitinol stent over 18 months while implanted in a juvenile sheep model.

FIG. 7 is a graph showing the thickness of a valve made with constructed tissue mounted inside a nitinol stent over 18 months while implanted in a juvenile sheep model.

FIG. 8 is a graph showing the maximum tension of a valve made with constructed tissue mounted inside a nitinol stent over 18 months while implanted in a juvenile sheep model.

### DETAILED DESCRIPTION OF THE INVENTION

A heart valve of the present invention is any heart valve known to one skilled in the art, with the added feature that the heart valve includes one or more constructed tissues of the present invention (as described in more detail below). Exemplary components made using tissue of the present invention include but are not limited to leaflet(s) and skirts.

In some embodiments of the invention, the valves include but are not limited to surgical valves; transcatheter valves; valves with stents, supports, and/or frames; frameless valves; valve assemblies with or without a skirt; arterial valves; and venous valves.

A tissue of the present invention is a non-fixed acellular biological matrix; may exhibit a burst pressure above 2000 mmHg, typically above 3000 mmHg; suture retention above 200 grams force; is allogenic and non-immunogenic; all of the above; or at least one of the above.

Embodiments of the present invention include artificial heart valves and their methods of use. In one embodiment, an implantable artificial heart valve includes a frame having a longitudinal axis extending between an inflow end of the frame and an outflow end of the frame, the inflow end of the frame being configured to receive antegrade blood flowing into the prosthetic valve when implanted, and a leaflet structure positioned within the frame and constructed of a constructed tissue. In some embodiments of the invention, the heart valve optionally includes an inner skirt positioned around an inner surface of the frame and extending along the longitudinal axis. The inner skirt may be formed from constructed tissue of the present invention.

In another embodiment, the leaflet structure and inner skirt are constructed of the same constructed tissue of the present invention.

In another embodiment, an assembly for implanting an artificial heart valve in a patient's body includes a delivery apparatus having an elongated shaft and a radially expandable artificial heart valve adapted to be mounted on the shaft in a radially collapsed configuration for delivery into the body. The prosthetic heart valve may include a frame having an inflow end portion defining an inflow end of the frame that is configured to receive antegrade blood flow into the artificial heart valve when implanted, the frame also having an outflow end portion defining an outflow end of the frame opposite the inflow end of the frame. The prosthetic heart valve may also include a leaflet structure positioned within the frame, a skirt folded along a portion of an inner surface of the frame. The leaflet structure is constructed of a constructed tissue, and, in accordance with one embodiment of the present invention, the skirt is formed by folding a portion of the constructed tissue used to make the leaflets. In this embodiment of the invention, the leaflet structure and the skirt are formed from a unitary or single piece of constructed tissue.

In some embodiments of the invention, the leaflets are stitchless in the area that, in prior art designs frequently fail in part because of the load in the area of the leaflet (e.g., the belly) where stitches are present. These areas, typically the belly of the leaflet and the commissure points, are high stress areas of the valve.

In another embodiment, as illustrated in the Figures, one or more constructed tissue tubes are cut and shaped into a portion that becomes a leaflet, and wherein said one or more leaflets function as a valve. Another portion of the same constructed tissue may be folded to function as a skirt.

As used herein, recellularization refers to the repopulation or growth of cells and structures near the implant site aiming to reconstitute and recreate the natural tissue-specific function. In some embodiments of the invention recellularization includes the tissue growing, including somatic growth, with the patient.

As used herein, regeneration refers to the ability of living organisms to replace damaged or lost tissue with new cells, restoring their structure and function, that is, becoming living tissue. Regeneration includes repopulation leading to restoration of tissue and/or body functions, including but not limited to restoration of cells, other biological molecules, and biological structures, including acellular ECM scaffolds. Regeneration is the process of renewal, regrowth, or restoration of a tissue, organ, or organism after damage, injury, or disease.

As used herein, remodeling refers to the process by which the body adapts to and integrates an implanted medical device. This process involves the interaction between the implant and the surrounding tissues, which can lead to changes in the structure and composition of the tissue.

As used herein, the inventors define recellularization and remodeling as being within the definition of regeneration.

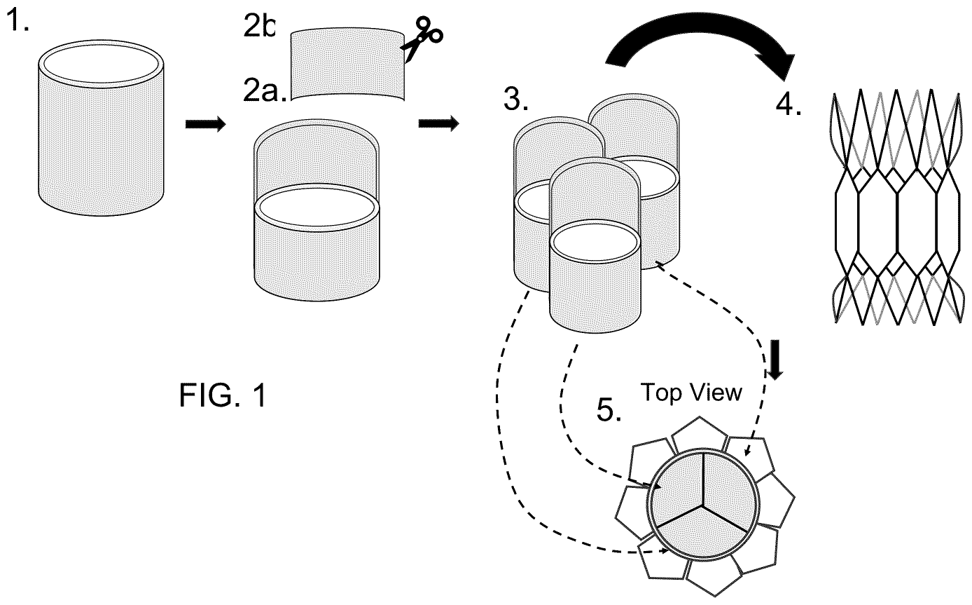

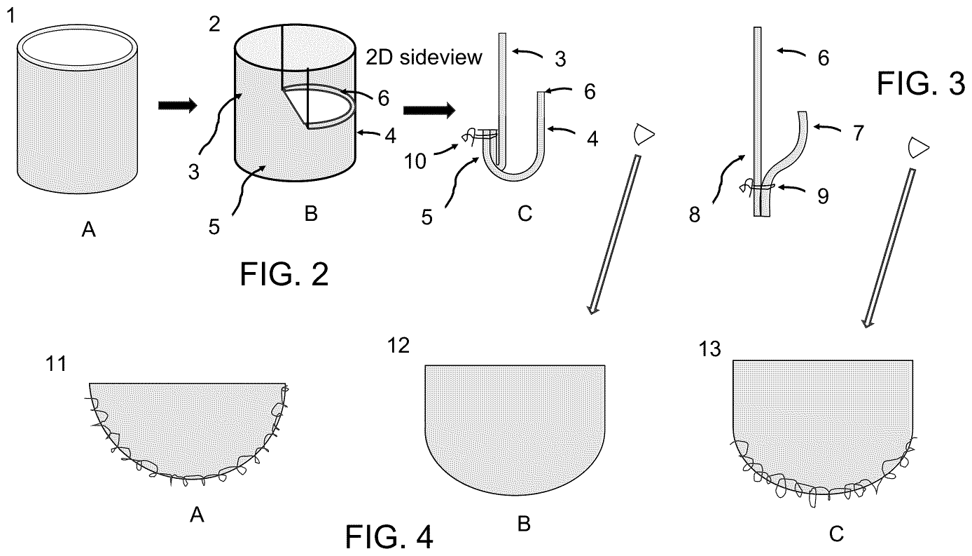

In an embodiment of the invention, one or more CT tubes, preferably three, are configured to form both a leaflet and a portion of the skirt. Each tube corresponds to a single leaflet and a portion of the skirt. Multiple tubes may be used together to form the complete skirt. Multiple tubes may be combined into a tube assembly, thereby forming a multi-leaflet valve. Other portions of tubes are folded to form the skirt. See for example, FIGS. 1 and 2C.

In accordance with the present invention, the tissue is folded in the belly region of the leaflet(s), i.e., contiguous to the annular region of the valve, near the inlet.

Folded, as used herein, includes folding up or down, toward the inlet side of the valve or the outlet side; and folding around the inlet end of the frame. In the preferred embodiment, a portion of the tissue is folded toward the outlet side, thereby forming a portion of the skirt.

An embodiment of the invention includes providing tube(s) of different diameters, sized according to the final diameter of the valve (formed, e.g., from multiple tubes). In this aspect of the invention, the valve is customizable in size to fit the native structure of the heart, including but not limited to providing adequate of sufficient leaflet coaptation.

In one representative embodiment, an implantable prosthetic valve comprises a radially collapsible and expandable frame or support; and a leaflet structure comprising a one or more of leaflets. Preferred embodiments include three leaflets. The leaflet structure is formed from the CT of the present invention and may be positioned inside of and secured to the frame. The valve may further include an annular skirt member, which can be disposed between the frame and the leaflet structure. In preferred embodiments of the invention, the leaflets and a portion of the skirt are formed from the same piece of constructed tissue, e.g., a portion of the tissue that forms a leaflet also forms a portion of skirt. As used herein, the inventors refer to this concept or structure as “contiguous.”

Another embodiment of the invention is a transcatheter valve wherein a single piece of constructed tissue is used to form both the leaflet (or valve assembly) and the skirt. In preferred embodiments of the invention, three tubes of constructed tissue are used, each tube having a portion used to form a leaflet and a portion used to form a portion of a skirt.

Another embodiment of the invention is a transcatheter valve formed from constructed tissue wherein a portion of the tissue is crimped inside a frame. In Examples 3 and 6, this is the first time that the tissue of the present invention has been crimped inside a frame and tested for long term mechanical and longevity properties.

Another embodiment of the invention is a transcatheter valve formed from constructed tissue and having the durability and mechanical properties sufficient for its intended purpose, e.g., a transcatheter pulmonary heart valve or a venous valve.

Another embodiment of the invention is a transcatheter valve wherein the stitches used to attach components of the valve to the frame are different than what is typically used in the prior art. FIG. 4A shows the typical stitch pattern for a pericardium tissue valve. FIG. 4C shows the stitch pattern for the valve described in Syedain, et al., Sci Trans Med 2021. FIG. 4B shows the stitch pattern for a valve of the present invention.

In another representative embodiment, an implantable prosthetic valve comprises a radially collapsible and expandable annular frame and a leaflet structure supported by the frame. The frame can comprise a plurality of interconnected struts defining a plurality of open cells. The valve is formed from constructed tissue (CT), and a portion of the tissue is crimped to the frame. The valve and frame are configured to be radially collapsible to a collapsed for introduction into the body on a delivery catheter; and radially expandable to an expanded state for implanting the valve at a desired location in the body (e.g., the native aortic valve). The frame can be made of any suitable material. Alternatively, the valve can be a so-called self-expanding valve wherein the frame is made of a self-expanding material such as Nitinol. A self-expanding valve can be collapsed to a smaller profile and held in the collapsed state with a restraining device such as a sheath covering the valve. When the valve is positioned at or near the target site, the restraining device is removed to allow the valve to self-expand to its expanded, functional size of open cells in the frame.

Suitable plastically-expandable materials that can be used to form the frame include, without limitation, stainless steel, a nickel-based alloy (e.g., a nickel-cobalt-chromium alloy), polymers, or combinations thereof.

Now referring to FIG. 2, a tube is made of the constructed tissue of the present invention (1A). A portion of the tube is cut away, and in 2C A portion 4 is formed into a leaflet and another portion 3 is formed into a skirt. Suture or stitch 10 connects the folded section of skirt to a frame (not shown). In FIG. 2C, element 6 is the leaflet tip and is sometimes referred to as the free edge of the leaflet. Element 5 refers to the folded portion of the skirt 3.

FIG. 3 shows a representation of a prior art valve 8, wherein element 7 is the leaflet, element 6 is the skirt, and element 9 is the suture or stitch used to connect both the leaflet and the skirt to a frame (not shown).

As noted above, the tissue of the present invention is folded to form a portion of the skirt. Folding the tissue makes the leaflet U-shaped (FIG. 4B) leaflet rather than classic C-shape (FIG. 4A).

An embodiment of the invention includes the direction in which the CT may be folded to form the skirt. I some embodiments, the tissue may be folded downstream of the valve. In a preferred embodiment, the tissue may be folded upstream of the valve, typically around or over the end of the frame, thereby providing a skirt or a portion of the skirt on the inflow side of the valve/leaflets or the inflow end of the implant/frame.

In another embodiment of the invention, the leaflet(s) and skirt are contiguous. In this embodiment, the leaflet and skirt are made from a single piece of tissue. In these embodiments, the skirt is first folded and stitched to the frame. Also in these embodiments, the leaflet belly is not sewn or stitched, and the valve assembly (multiple leaflets) does not need stitches encompassing the entire commissures and belly of each leaflet.

The skirt of a prosthetic valve serves several functions. In particular embodiments, for example, the skirt functions to seal and prevent (or decrease) perivalvular leakage, to anchor the leaflet structure to the frame, and to protect the leaflets against damage caused by contact with the frame during crimping and during working cycles of the valve.

In preferred embodiments of the invention, a tissue of the present invention recellularizes without evidence of degradation of the recipient tissue near the implant site

In the most preferred embodiments, the tissue of the present invention, when implanted, does not degrade. This feature is in contrast to products that have a synthetic or biological portion that is specifically degradable, e.g., a scaffold. This feature is also distinct from the tissue as it is being formed; during formation, the ECM-producing cells degrade fibrin until a collagenous tissue is formed. The cell-containing tissue contains no, very little, or not detectable amounts of fibrin.

In preferred embodiments of the invention, the histology analysis of the explanted tissue valve showed no or little immune response; no evidence of calcification; thin uniform leaflet thickness; endothelial cells covering the leaflet surfaces; evidence of elastin fibers in neo-tissue; and neo-tissue composed of interstitial cells.

The inventors believe the ability of the constructed tissue to not degrade contrasts with what one skilled in the art would expect. The inventors showed photographs of the tissue to one skilled in the art (a Board-certified pathologist), and that person indicated that it was not possible without degradation of the tissue near the implant. This person has cited publications supporting their position, that one skilled in the art would expect degradation to precede recellularization for all known tissues.

Therefore, an embodiment of the invention includes but is not limited to a tissue of the present invention that recellularizes without first degrading the tissue near and around the implant site.

Another embodiment of the invention includes but is not limited to a tissue of the present invention that recellularizes without itself degrading, e.g., structural or scaffold degradation as part of the tissue synthesis process. In preferred embodiments of the invention, the tissue of the present invention is a non-immunogenic regenerative acellular allograft.

An embodiment of the invention includes a product or implant formed from the tissue of the present invention.

An embodiment of the invention includes other implants that may be covered or wrapped in whole or in part with a tissue of the present invention. In these embodiments, the CT prevents or reduces an immune response or infection from the underlying implant.

Embodiments of the invention include but are not limited to the form of tissue delivery. Examples include endovascular delivery and surgical implant.

The implant of the present invention may be used in a method of treating a patient having a wide variety of conditions, diseases, or injuries, the common theme of which is that the treatment involves an implant that includes a tissue in whole or in part.

In preferred embodiments of the invention, the tissue of the present invention as an implant was tested for its risk to a patient, specifically patients with pre-existing conditions and underlying conditions. It was found that the tissue of the present invention caused little or no risk to the patient, and in some cases, was beneficial to the patient in treating implant site infections not caused by the implant.

The tissue of the present invention may be any size or shape.

The present invention is an implant, prosthesis, or covering formed from constructed (CT) regenerative, and/or engineered tissue. As used herein, constructed or engineered refers to the fact that the inventors and others may produce or construct the tissue, e.g., the tissue is not a product of nature. In preferred embodiments of the invention, the tissue mediates regeneration without causing degradation of tissue and other biological material in the area of the implant site. The invention includes methods of making the tissue and methods of making the graft or prosthesis.

The tissue may be formed by combining ECM-producing cells in the presence of fibrinogen and thrombin under conditions that permit the formation of regenerative tissue. An exemplary process is shown in FIG. 1. Typically, the process involves forming a cell-seeded suspension comprising ECM producing cells, fibrinogen, and thrombin. The suspension is then cast over a form and allowed to incubate. During incubation, an ECM/fibrin/collagen tissue begins to form. As part of that process, compaction and fiber alignment occurs, leading to remodeling of the ECM/collagen/fibrin tissue as it forms. The tissue is then cultured until it matures, e.g., is substantial enough to be used for its intended purpose. The resulting cell-containing tissue is then decellularized. Some embodiments of the tissue have been previously described. The tissue of the present invention is cultured from completely biological raw materials and allogeneic dermal cells. For example, see the patents and patent applications listed below.

In preferred embodiments of the invention, the hydrogel-ingredients in a suspension-allows the tissue to grow in a volumetric 3-D process also known as casting. In contrast, most if not all typical tissue engineering methods use a synthetic and/or immunogenic scaffold or the like to grow the tissue in a 2-D manner (cell suspension seeded on the surface). The growth eventually produces a 3-D construct, but the growth in scaffold-based constructs is different that the volumetric 3-D casting growth in tissues of the present invention. In many prior art 2-D processes, the scaffold degrades, leaving some type of tissue or cellular matrix.

A preferred embodiment of the invention is any structure or shape formed from the tissue of the present invention, including but not limited to a tubular graft.

In accordance with embodiments of the present invention, any prosthesis may be formed in whole or in part using regenerative tissue (RT) or engineered tissue. RT, as used herein, refers to tissue formed or processed as disclosed in the following: 2007/061800; WO 2007/092902; 2016/0203262; WO/ 2004/018008; WO 2004/101012; PCT/US21/62709 (filed 9 Dec. 2021); PCT/US2017/026204 (filed 5 Apr. 2017); U.S. Pat. Nos. 10,111,740; 10,105,208; 10,893,928; 8,192,981; 8,399,243; 8,617,237; 8,636,793; 9,034,333; and 9,126,199; U.S. Ser. No. 17/139,575 filed Dec. 31, 2020 (issue fee paid); U.S. Ser. No. 16/500,147 filed Oct. 2, 2019 (issue fee paid); U.S. Ser. No. 10/523,618; U.S. Ser. No. 10/556,959; U.S. Ser. No. 13/771,676; 2015/0012083; 2009/0319003; 2011/0020271; 2012/0230950; 2013/0013083; 2014/0330377; 2014/035805; 2017/0135805; 2017/0296323; 2017/0306292; U.S. Pat. Nos. 8,198,245; 9,127,242; 9,556,414; 9,657,265; and 9,650,603; all of which are hereby incorporated in the entirety be reference.

In one embodiment of the invention, the bioengineered tissue may be made according to U.S. Pat. Nos. 10,111,740; 10,105,208; 10,893,928; and 11,589,982, all Tranquillo, et al., each incorporated in its entirety be reference. Any process or method for producing engineered tissue involving ECM-producing cells in a hydrogel is included within the scope of the present invention.

The CT of the present invention, may be characterized by lack of evidence of patient infection (in vivo); lack of evidence of patient immune response (in vivo); lack of evidence of toxicity; lack of evidence of implanted tissue degradation; lack of evidence of residual cellular debris (e.g., particle shedding from the tissue, in contrast to polymer degradation and erosion); modified (e.g., reduce or eliminate) inflammation, calcification characteristics, resorbability, suture retention, size and shape, thinness (e.g. dilatation or aneurysm formation), collagen content, and other characteristics and properties that will become clear from the description of the invention.

In another embodiment, the constructed tissue of the present invention includes collagen types I, III, and VI; tenascin; and fibronectin.

The methods, uses, and products of the present invention are intended for implant in a mammal, preferably but not limited to a human.

A product and/or method of the present invention typically includes combining fibrinogen or fibrinogen-like material, thrombin, and matrix-producing cells to produce a fibrin gel with a homogeneous cell suspension. In preferred embodiments of the invention, the cell infused fibrin gel undergoes casting, used herein to refer to encapsulating cells in a fibrin gel, and culturing to form the collagenous tissue or grafts. Casting may occur with or without a form or mold. In other preferred embodiments, the tissue or graft may be allowed to contract (e.g., longitudinally or radially), preferably in a controlled manner. In accordance with the present invention, the process permits customized or optimized fiber alignment during the contraction phase. Customized or optimized alignment includes, but is not limited to radial alignment, longitudinal alignment, both radial and longitudinal alignment, and a pre-determined ratio of radial and longitudinal alignment

The CT of the present invention is distinct from certain other kinds of regenerative or engineered tissue in the use of completely biological raw materials and allogeneic dermal cells; and in the use of crosslinked fibrinogen that is later degraded during the culturing process. Also, the CT of the present invention can be contracted or allowed to contract, for example, in the longitudinal direction and/or in the radial direction, among others. In accordance with some embodiments of the invention, the fibers in the tissue may align or become aligned, believed to be partially due to fibrin having no or little resistance to contraction that occurs naturally as part of the collagen/ECM formation process. The inventors also believe that radial and/or longitudinal contraction occurs in part naturally as an inherent function of tissue forming as described herein. In another embodiment of the invention, the contraction may be scalable or intentionally controlled to enhance, promote, or achieve one or more tissue characteristics, e.g., fiber alignment, or tensile strength, or suturability. Furthermore, the CT of the present invention does not include any synthetic materials, as is typical in other processes that use PLA or PGA or the like.

One or more methods of the present invention may also include molding or forming the cell-seeded fibrin gel into a pre-determined shape; manipulating, mechanically and/or manually, the growing tissue in the presence of culture medium to produce CT; manipulating the growing tissue during the culturing phase of the tissue; manipulating the tissue during the maturation phase of the tissue; manipulating the tissue during the culturing/maturation phase of the tissue production process; manually moving the growing tissue to evenly distribute the stress relief from the contracting ends; decellularizing the CT; and automated or semi-automated versions of any of the method steps.

The CT of the present invention may be characterized by one or more of the following: non-oriented fibers; oriented fibers; thickness up to about 2 mm, preferably between about 100 μm and about 800 μm; diameters greater than about 1 mm; diameters from about 1 mm to about 40 mm, preferably from about 2 mm to about 25 mm, most preferably from about 3 mm to about 16 mm; lengths greater than about 1 cm; lengths from about 1 cm to about 100 cm, preferably 10 cm to 30 cm, and most preferably about 12 cm to about 22 cm; non-immunogenic or minimally immunogenic; a tissue, sheet or shape that is anisotropic; a tissue, sheet, or shaped structure produced by a process that includes scaled contraction (as described above); a sheet or shape that is suitable for cutting into shapes, e.g., by scalpel, die, or laser; suppleness; suturability; no or little calcification during life of implant; crosslink density, or variations of crosslink density through the material thickness; collagen concentration; collagen density, or variation of crosslink density through the material thickness; remodeling proclivity; absorption; resorption; degradability, regions of greater stiffness; regions of greater flexibility.

In preferred embodiments of the invention, by controlling compaction as the tissue matures during formation, the thickness of the tissue may be varied.

In preferred embodiments of the invention, the RT has oriented fibers leading to suture pull-out resistance, anisotropic material properties as witnessed by tensile strength, even more preferably, adapted for its end use (e.g., a sheet, or a tube, or a valve).

In another embodiment, the delivery apparatus is a catheter.

In another further embodiment, the catheter is a self-expanding frame, preferably with shape memory.

In an alternative embodiment, the catheter may be a balloon catheter including a balloon, the balloon is deflated, the radially expandable artificial heart valve is positioned over the balloon, and the delivering step is accomplished by inflating the balloon, the inflating balloon radially expands the radially expandable artificial heart valve.

A further embodiment includes a method of treating a patient for a valvular disease including identifying a valvular disease in a patient, and implanting an artificial heart valve into a blood vessel of the patient, wherein the leaflet structure is constructed of a constructed tissue, and the inner skirt is constructed of a constructed tissue, the same or different from the tissue used to form the leaflets.

### Various Method Steps

Some embodiments include forming one or more tubes or cylinders of constructed tissue of the present invention, removing a portion of the tube, using a first portion of the tube as a leaflet, and folding another portion of the tube to form a skirt.

By mimicking the extra cellular matrix of the natural environment, a tissue can be grown having desirable or beneficial structural properties, which eventually develop towards a native-like architecture (i.e., the tissues of the present invention are a biomimetic material). In preferred embodiments of the invention, the tissue of the present invention may be handled in a similar way like a native vein or artery when surgically implanted.

Some embodiments of the invention may further include storing and/or sterilizing a medical device or tissue of the present invention. These embodiments may include preselected storage solution; preselected sterilization solution or technique; storage packaging; and/or sterilization packaging. In one embodiment, the tissue may be stored in PBS and refrigerated until use. In another embodiment, the tissue may be partially or fully dehydrated. In one embodiment, the storage is in a sterile dry container. Other storage/sterilization processes may include one or more additives known to those with skill in the art. In another embodiment, the tissue may be E-beam sterilized in PBS alone.

One skilled in the art will recognize that other storage and sterilization protocols may be used with the tissue of the present invention.

In some embodiments of the invention, the tissue exhibited greater cellularization. For example, in comparing the tissue of the present invention to published descriptions of other tissues (Lawson et al, Lancet, 2016, Vol. 387, page 2031), the tissues of the present invention exhibited greater cellularization (faster cellularization over time). The present invention is recellularized with smooth muscle actin-positive cells across the entire thickness.

In some embodiments of the invention, the tissue exhibited recellularization where it was not expected to be. For example, Lawson shows sparse smooth muscle actin positive cells near the human surface of their tissue. In contrast, the tissue of the present invention exhibits consistent presence of smooth muscle actin positive cells across the thickness of implanted tissue.

In some embodiments of the invention, the tissue when implanted in humans did not degrade prior to recellularization or body cell infiltration.

### Other Embodiments

It should be noted that various embodiments of artificial valves and systems for delivery and implant are disclosed herein, and any combination of these options may be made unless specifically excluded. Likewise, the different constructions of artificial valves may be mixed and matched, such as by combining any valve type and/or feature, tissue cover, etc., even if not explicitly disclosed. In short, individual components of the disclosed systems may be combined unless mutually exclusive or otherwise physically impossible.

In various embodiments, the tissue utilized for the inner skirt and leaflet structure, including leaflets, is regenerative tissue, such that the artificial valve will integrate into the body of the individual receiving the artificial valve. Suitable materials will allow the patient's body to fully integrate the material, such that the material will continue growing with the body of the patient. Such material will allow the valvular structure and skirt to grow in a concomitant manner as the patient's heart grows such that replacement is not required. Regenerative materials may include decellularized tissue from a natural source, which may require ligation of branching blood vessels. Examples of regenerative tissue and methods of constructing these materials can be found in PCT/US2021/62709, the disclosure of which is incorporated herein by reference in its entirety.

A tissue product of the present invention be unsupported or may further include a support member, such as a nitinol support or scaffold. The support member may be internal, external, or embedded in the tissue.

In some embodiments of the invention, the implanted tissue, graft, or implant is capable of endothelialization, even substantial endothelialization, features that provide evidence of the long-term biocompatibility of the tissue and mediation in vascular repair.

In preferred embodiments of the invention, CT tissue grafts have been shown to provide anatomical and functional characteristics that mimic native structures, e.g., native vessels around the heart and saphenous veins.

In preferred embodiments of the invention the CT tissue were evaluated for its mechanical properties and hemodynamics. All of these evaluations showed that the CT tissue of the present invention has similar compliance and “hand” to native structures.

The present invention also is a surgical kit comprising one or more of the following: a regenerative tissue implant or graft processed or produced according to the present invention; one or more instruments for implanting the graft; a rinse tray; a rinse solution, e.g., heparin; and suture material.

One of the embodiments of the present invention is a sterile closed package containing a biological material of the present invention. Typically, a separate container would hold individual or multiple samples having known size or dimensions. If desired, the biological material in the sterile package can be attached to another material or structure, such as an annuloplasty ring, a sewing cuff, a synthetic graft, or a support for positioning the biological material on a stapler.

An implant of the present invention may be delivered in any medically acceptable manner. In a preferred embodiment, the tissue is surgically delivered. In another embodiment, the tissue is delivered via catheter or tube.

One skilled in the art will recognize that the processes steps described herein may be variously modified and a wide variety of ways in order to achieve a tissue with certain properties, according to present invention.

### Definitions

The following definitions are used in reference to the invention:

As indicated herein, a decellularized vessel consists essentially of the extracellular matrix (ECM) components of the vascular tree. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei in histologic sections using standard histological staining procedures. Preferably, but not necessarily, residual cell debris also has been removed from the decellularized organ or tissue.

(B) As used herein, biomimetics or biomimicry refer to imitating the models, systems, and elements of nature for the purpose of solving complex human or animal problems. In the present invention, biomimetics is used for therapeutic purposes.

### REFERENCES

Syedain Z H, Graham M L, Dunn T B, O'Brien T, Johnson S L, Schumacher R J and Tranquillo R T. A completely biological “off-the-shelf” arteriovenous graft that recellularizes in baboons. Sci Transl Med. 2017; 9.

### EXAMPLES

### Example 1

A tissue of the present invention was implanted as a CABG graft into an ovine model, and after six months showed long-term performance and regeneration into a living blood vessel.

A tissue of the present invention was implanted as a vascular patch into an ovine model, and after six months showed appropriate performance, lack of calcification, and no sign of infection.

A tissue of the present invention was implanted as a pediatric vascular conduit into a lamb model, and after 18 months showed somatic growth and regeneration.

A tissue of the present invention was implanted as a pulmonary valve conduit into a lamb model, and after one year showed somatic growth and lack of calcification.

A tissue of the present invention was implanted as an aortic frameless valve into an adult sheep model, and after six months showed durability and normal hemodynamics.

A tissue of the present invention was implanted as a transcatheter pulmonary valve into a juvenile sheep model, and after 18 months showed durability and normal hemodynamics, and lack of calcification.

### Example 2

Histological analysis for regenerative tissue potency, baboon v. human, histological pics comparing ovine, baboon, and human, SMA stain, first in man (FIM) biopsy, endothelial stain, microvascular on adventitial layer.

Examples of recellularization of constructed tissue implanted in ovine, baboon, and human subjects show recellularization with smooth muscle actin positive cells and capillary formation.

### Example 3

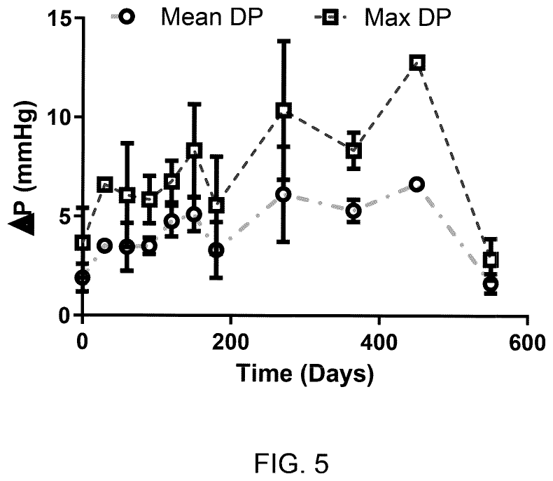

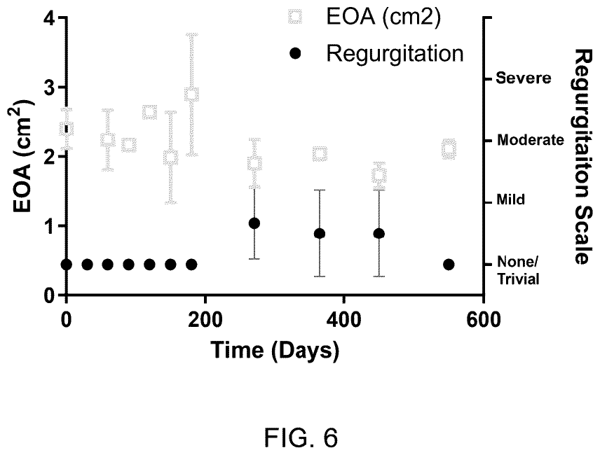

The valve made with constructed tissue mounted inside a nitinol stent was implanted as pulmonary valve replacement in juvenile sheep model. The hemodynamics properties of valve function were monitored over the course of implant for eighteen months. During the implant duration, no change was observed in systolic pressure drop as measured by both mean and peak pressure drop. See FIG. 5. Further, the effective orifice area stayed stable between 2-3 cm². The regurgitation monitored for the course of implanted stayed at trivial to mild. See FIG. 6.

### Example 4

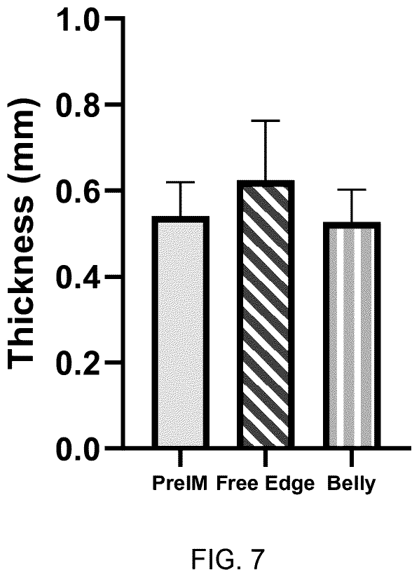

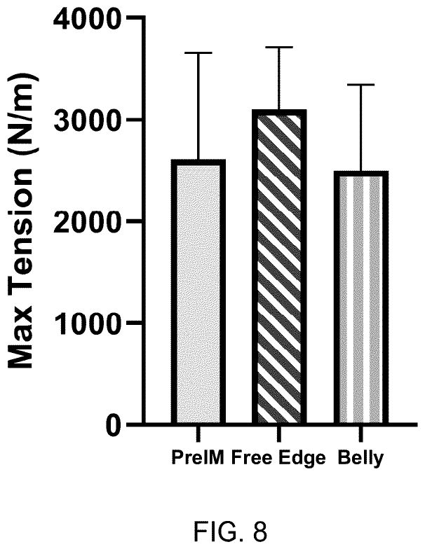

A tissue of the present invention was tested for various mechanical properties before and after the heart valve was implanted for eighteen months. The thickness measured in millimeters (see FIG. 7) and maximum tension measured in newton per meter (see FIG. 8) showed no change between pre-implant and explanted tissue. The explanted tissue was measured in belly of the leaflet and free-edge region of the leaflet.

### Example 5

A tissue of the present invention was tested for various mechanical properties before and after implanting. The mechanical properties of the post-implant tissue showed no decrease in any biologically critical property.

### Example 6

A tissue of the present invention was tested for calcification before and after implanting as heart valve for eighteen months in juvenile sheep model. Quantitative calcium content measurement performed using Inductively Coupled Plasma Mass Spectrometry measured pre implant average value of 0.218 mg/mg dry tissue, with measurements in two valve explants had average of 0.151 mg/mg dry tissue and 0.241 mg/mg dry tissue.

### Example 7

A pre-clinical evaluation of the CT tissue was conducted in a patient that was infected during the surgical implant process. The evaluation showed that the implant was not the cause of the infection and in fact aided in reducing the infection.

### Example 8

In one embodiment, illustrated in FIG. 1, the constructed tissue made in the form of 16 mm diameter tube is cut into 3 cm length segments (1). Three of these selected tubes are further modified to remove a flap (2b), equivalent to half of the circumference of the tube (2a). 2C shows the side view of a single tube and how a portion of the tissue becomes leaflet 4 and another portion is folded to become a portion of skirt 3. In an exemplary transcatheter pulmonary valve, FIG. 1 (3) shows three tubes (3) inserted inside a support structure made of metal (4) to produce the final valve with skirt 5A. In one embodiment, the metal is nitinol. For each tube, the side with full length faces the support structure wall encompassing one third of the circumference of the support structure. This segment of the tube when anchored to the support structure via stitching (FIG. 2(C), element 10) forms the skirt segment of the valve design. The shorter length segment of constructed tissue forms the leaflet; three leaflets are formed when using three tubes. The bottom of each tube is closed via stitching or fold, thus creating a pocket inside each tube (FIGS. 1, 5a&b; FIG. 2C). During function as a heart valve, with forward flow, the flow of fluid collapses each tube onto itself thus creating a central orifice for fluid to flow through. During reverse flow, each tube forms a pocket covering one third of the lumen area of the constructed valve, thus closing the entire orifice leading to no back flow. Overall, the design works as a one-way valve.

### Example 9

In vitro durability testing demonstrated that stitch line tear after 50-70 million cycles showing weakness as durable closure for belly of leaflet.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

While the invention has been described in some detail by way of illustration and example, it should be understood that the invention is susceptible to various modifications and alternative forms and is not restricted to the specific embodiments set forth in the Examples. It should be understood that these specific embodiments are not intended to limit the invention but, on the contrary, the intention is to cover all modifications, equivalents, and alternatives falling within the spirit and scope of the invention.

1. An implantable transcatheter valve comprising constructed tissue formed by combining thrombin, fibrinogen, and extracorporeal matrix-producing cells; wherein said constructed tissue, when introduced in vivo into a subject, induces recellularization in vivo.

2. The valve of claim 1 wherein said tissue further comprises a non-fixed acellular biological matrix, has a tensile strength above 2 MPa (mega Pascals), has suture retention strength above 200 grams-force, and is non-immunogenic.

3. The valve of claim 1 wherein said tissue is durable to at least 200 million cycles.

4. The valve of claim 1 wherein said valve is a heart valve.

5. The heart valve of claim 4 wherein said heart valve is a transcatheter pulmonary heart valve.

6. The valve of claim 1 further comprising wherein a portion of said tissue forms one or more leaflets.

7. The valve of claim 6 wherein the belly of a leaflet is sutureless.

8. The valve of claim 7 further comprising wherein the commissure is sutureless.

9. The valve of claim 1 further comprising wherein said tissue is folded to form a skirt.

10. The valve of claim 1 wherein said tissue mediates natural processes including remodeling and/or regeneration.

11. The valve of claim 10 wherein natural processes includes elastin deposition in vivo.

12. The valve of claim 1 further comprising:
a radially collapsible and self-expandable annular frame, a first row of circumferential tapered diameter struts defining an inflow end of the frame, a second row of circumferential struts spaced apart from the first row adjacent having uniform and smaller diameter, and a third row of circumferential struts spaced apart from the second row adjacent defining a tapered larger diameter outflow end of the frame, and
a leaflet structure comprising at least one leaflet formed from the constructed tissue.

13. The valve of claim 12, further comprising an annular skirt member positioned between the annular frame and the leaflet structure, said skirt being formed by folding a portion of said constructed tissue.

14. The valve of claim 12 wherein each of said leaflets is formed from a tube of said constructed tissue.

15. The valve of claim 1 wherein said valve is suitable for implanting in any tubular anatomy of a mammal.
